(19) United States
(12) Patent Application Publication
(10) Pub. No.: US 2021/0077753 A1
(43) Pub. Date: Mar. 18, 2021

(54) NEBULIZER DELIVERY SYSTEMS AND METHODS

(71) Applicant: BN Intellectual Properties, Inc., St. Petersburg, FL (US)

(72) Inventors: Donald M. PELL, St. Petersburg, FL (US); Michael P. SPUZA, St. Petersburg, FL (US); Govindan P. NAIR, St. Petersburg, FL (US); Mark J. HOYT, Midvale, UT (US); Chad S FRAMPTON, American Fork, UT (US); Kent MABEY, West Jordan, UT (US)

(21) Appl. No.: 17/100,566

(22) Filed: Nov. 20, 2020

Related U.S. Application Data
(63) Continuation-in-part of application No. 16/836,485, filed on Mar. 31, 2020.
(60) Provisional application No. 62/827,604, filed on Apr. 1, 2019.

(51) Int. Cl. A61M 15/00; A61M 11/00

(52) U.S. Cl. CPC A61M 15/009; A61M 15/0021; A61M 15/0085; A61M 15/0086; A61M 15/0066; A61M 2205/3592; A61M 2205/3324; A61M 2205/18; A61M 2205/52; A61M 2205/609; A61M 11/005

(57) ABSTRACT

A method of using a nebulizer includes connecting a medicine vial containing a medicine solution to the nebulizer and reading a medicine conductivity and/or pH value from the medicine vial. The conductivity and/or pH of the medicine solution is measured and compared with the medicine conductivity and/or pH value. When the medicine conductivity and/or pH value and the measured conductivity and/or pH of the medicine solution match, the flow rate value and dosage timings are read from the medicine vial; and the mesh is activated at the medicine flow rate value to produce a plume of particles of a medicine solution at the beginning of an inhalation. The active mesh is deactivated to stop making particles by manually or at a calculated time. The activation of the active mesh is restricted to approved users by means of an authentication code or biometric feature information.

### PRIORITY CLAIM

The present disclosure claims priority to U.S. patent application Ser. No. 16/836,485 filed on Mar. 31, 2020 and U.S. Patent Application 62/827,604 filed on Apr. 1, 2019, the contents of which are incorporated herein by reference in its entirety.

### BACKGROUND

Nebulizers deliver pharmacological products to a user by generating small droplets from a solution of the pharmacological product, which are inhaled into the lungs for treatment of medical conditions.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic diagram of an active mesh nebulizer, in accordance with some embodiments.

FIG. 2 is a flow diagram of a method of operating an active mesh nebulizer, in accordance with some embodiments.

FIG. 3 is a diagram of a dosing schedule, in accordance with some embodiments.

FIG. 4 is a chart of particle sizes in a plume of particles, in accordance with some embodiments.

FIG. 5 is a table of particle size data for a plume of particles, in accordance with some embodiments.

FIGS. 6A and 6B are diagrams of a vial assembly for an active mesh nebulizer, in accordance with some embodiments.

FIGS. 7A and 7B are diagrams of an active mesh nebulizer, in accordance with some embodiments.

FIG. 8 is a flow diagram of a method of operating an active mesh nebulizer, in accordance with some embodiments.

FIG. 9 is a flow diagram of a method of operating an active mesh nebulizer, in accordance with some embodiments.

FIG. 10 is a diagram of a nebulizer mouthpiece baseplate, in accordance with some embodiments

FIG. 11 is a flow diagram of a method of operating an active mesh, in accordance with some embodiments.

FIG. 12 is a diagram of an active mesh with an adjustable mesh voltage, in accordance with some embodiments.

FIG. 13 is a block diagram of an active mesh nebulizer, in accordance with some embodiments.

FIG. 14 is a flow diagram of a method of operating an active mesh nebulizer with an adjustable mesh voltage, in accordance with some embodiments.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, etc., are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In a medical setting, nebulizers are used to deliver pharmacological compounds to medical patients for treatment of medical conditions. Nebulizers are also used to deliver non-medical products to persons in non-medical settings, such as nicotine to persons in nicotine-replacement therapy. However, previous nebulizers, including aerosol, passive mesh, and active mesh nebulizers, exhibit imprecise dosage control of the pharmacological compound being supplied to the patient. In many instances, nebulizers volatilize a solution having one or more pharmacological compounds into droplets and the dosing of the patient or user is regulated by the amount of time that the user spends inhaling the stream of particles or droplets, and/or the efficiency with which the stream of particles or droplets is directed toward the patient or user's nose or mouth to be inhaled. In many situations, patients are treated with incorrect dosage of a pharmacological compound in order to achieve a rapid response in the patient's body, which sometimes results in side effects from the over dosage of the pharmacological compound.

Treatment of a patient or user with open cup nebulizers involves placing a solution of the pharmacological compound in a bowl or cup, directing a flow of air through the solution to generate particles or droplets of the solution, and the flow of air directs the particles or droplets toward the patient or user's nose and/or mouth for inhalation. Open cup nebulizers produce a constant stream of particles or droplets which are inhaled at will by the patient or user.

HFA (hydrofluoroalkane) inhalers provide a more accurate dosage regimen than open cup nebulizers for a patient or user, where a pharmacological compound in suspension and a propellant are expelled from an inhaler mouthpiece in a high velocity stream of liquid and expanding gas into a patient or user's mouth during an inhalation. HFA inhalers do not reliably provide accurate dosages of pharmacological compounds to a patient or user. When a patient does not agitate the suspension of pharmacological compound in the HFA inhaler, the amount of pharmacological compound delivered to a user in a metered spray is below an anticipated level of the pharmacological compound because of insufficient mixing. Also, the direction of a stream of suspension into the patient or user's mouth is difficult to control. When the stream strikes the tongue, cheeks, or throat of the patient or user, the liquid tends to adhere to the tissue rather than continue into the lungs, reducing the effectiveness of dosing a medical condition with HFA inhalers. Patients or users also tend to cough when the suspension strikes the upper portions of the respiratory tract, expelling some of the suspension and further reducing the amount of pharmacological compound retained or absorbed by a patient or user. Thus, accurate dosage of medical conditions with HFA inhalers is difficult to coordinate. One or more embodiments of the present disclosure describe an active mesh nebulizer configured to provide accurate dosing of solutions of pharmacological compounds to patients or users.

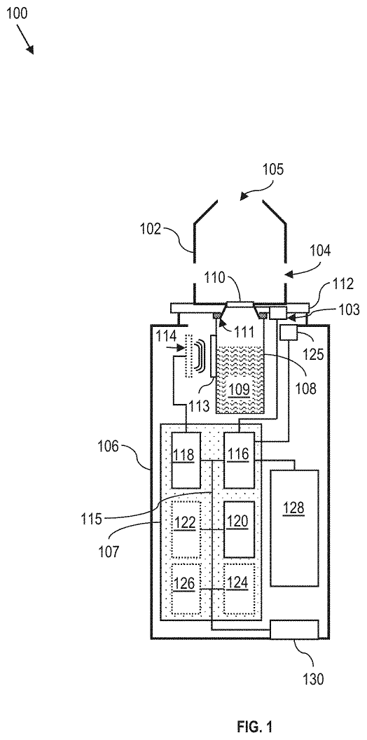

FIG. 1 is a schematic diagram of an active mesh nebulizer 100, in accordance with some embodiments. An active mesh nebulizer is a nebulizer which produces a plume of particles or small droplets by causing an active mesh 110 to vibrate. The active mesh nebulizer 100 includes a mouthpiece 102 with at least one hole 104 therein to allow air to enter the mouthpiece during a patient inhalation during operation of the active mesh nebulizer to produce a plume of particles (droplets) to treat a patient medical condition. In at least some embodiments, the at least one hole 104 is positioned on a side of the mouthpiece 102 and is configured to direct entering air toward the plume of particles and promote particle transport through a mouthpiece opening 105 and thereby into the patient's lungs. The mouthpiece 102 fits against a nebulizer body 106.

In some embodiments, active mesh nebulizer 100 includes a sensor 125 to detect the mouthpiece 102 being against the nebulizer body, and/or the closure of the nebulizer body 106 with a vial 108 located therein. In some embodiments, the nebulizer body 106 and mouthpiece 102 are integral, and the vial 108 is added to the nebulizer from an opening in the nebulizer body, where a sensor monitors the body closure. The sensor is configured to monitor when the nebulizer body is opened and/or closed to ensure that the vial 108 containing a solution of pharmacological compound is not removed, substituted, or tampered with. Such monitoring, and ensuring that the vial assembly is not removed, substituted, or tampered with, is one aspect of securely providing pharmacological compounds to patients or users within medically acceptable dosing limits. Adulteration of pharmacological compound solutions is to be avoided because the nebulizer disclosed herein is more efficient than other approaches at providing pharmacological compounds to users via inhalation of plumes of particles or droplets. In some embodiments, the vial is made of glass in order to safely hold pharmaceutical compounds or medicinal compounds. In some embodiments, the vial is made of an organic or polymeric material. An organic or polymeric material is suitable for holding compounds that are for purposes other than treating medical conditions.

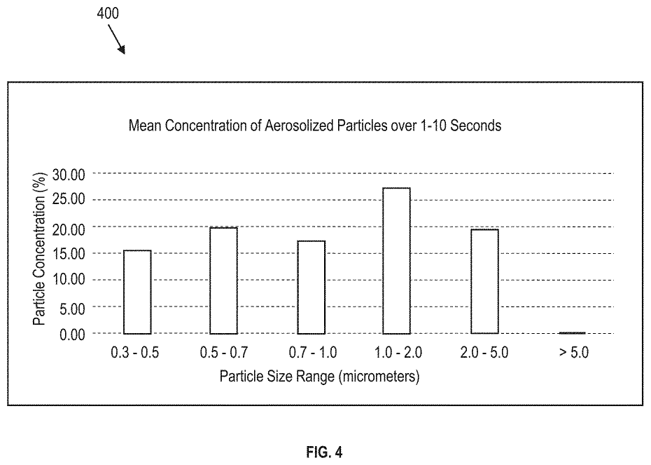

An active mesh 110 produces a plume of particles or droplets (not shown) that is directed toward a patient or user's lungs during inhalation by the user. Active mesh 110 is configured to produce particles having a diameter of not greater than 10 micrometers (μm). In some embodiments, more than 99% of the particles (or droplets) of solution in a vial in the nebulizer produced by the active mesh 110 have a mean particle diameter of 10 micrometers or less (see FIG. 4). In some embodiments, more than 95% of the particles or droplets produced by the active mesh 110 have a mean particle diameter of 5 micrometers or less. The subject matter of the present disclosure is extendable to nebulizers that produce plumes of particles or droplets with a wide range of particle distributions that also produce particles having diameters below 10 micrometers. An active mesh nebulizer produces, from a solution in the vial of the nebulizer, a plume of particles as a result of the mesh vibration, rather than by heating or boiling the solution. Thus, there is no contamination of the solution with mesh material, as occurs when a metallic heating element is used to elevate the temperature of a solution to produce vapor or streams of particles (e.g., in many e-cigarette devices). Further, by producing a plume of particles without heating or boiling the solution, there are no chemical changes to the pharmacological compounds of the solution because of elevated temperature during delivery to a patient or user.

The ability of particles to penetrate into the lungs and be absorbed by the body is a function of the size of the particles and the respiratory pattern of the user. Inhaled particles having a diameter greater than about 15 micrometers penetrate into the lungs as far as the bronchi because the cilia of the lungs capture the inhalable particles from further travel into the lung volume. Some small amount of the particles are absorbed, while most particles are cleared by the cilia and swallowed by the user after inhalation. Thoracic particles, ranging in size from 10 to 15 micrometers, penetrate into terminal bronchioles in the lungs. Particles ranging in size from 0.1 to about 6 micrometers are able to penetrate into the alveoli in the lungs and are readily absorbed through the alveoli into the circulatory system and body tissues. Particles that are unable to penetrate into the alveoli are absorbed into lung tissue and into the bloodstream with lower efficiency than particles that reach the alveoli and which are absorbed directly into the bloodstream across alveolar membranes.

Open pot nebulizers do not provide accurate doses because the patient inhalation time and volume of inhaled pharmaceutical product is extremely variable, depending on a patient's choice for inhalation duration and the amount of leakage of particles outside of the patient's mouth.

The absorption of the plume of particles or droplets increases when a patient or user of an active mesh nebulizer employs deep, slow inhalation for entrainment of the particles or droplets into alveolar spaces of the lungs. In some embodiments, the patient or user performs an inspiratory action over the course of 2-8 seconds and holds the inspired particles or droplets within the lungs to further promote absorption of the particles. In a preferred embodiment, the inhalation period or inspiratory action lasts between 3 and 6 seconds. In a preferred embodiment, the patient or user holds the plume of particles in the lungs for at least 5 seconds before an exhalation of the air from the lungs. According to some embodiments, the pause between inspiratory actions (and corresponding plume generation) is regular and even. In some embodiments, the patient or user regulates the duration of the pause between inspiratory actions and/or plume generation. In some embodiments, the duration of the pause between inspiratory actions and/or plume generation is regulated by the nebulizer, or by a third party such as a health-care professional that programs the nebulizer.

Active mesh 110 produces a plume of particles by vibrating at high frequency to trigger particle, or droplet, formation from a liquid against an inner surface of the mesh (e.g., the side facing the interior of the vial in the vial 108) on an outer surface of the mesh (e.g., the side facing the mouthpiece interior volume and mouthpiece opening). Active mesh 110 is a disc having openings extending through the planar surface of the disc, such that the disc, when electrically stimulated to undergo piezoelectric vibration, oscillates against a solution 109 in the vial 108, causing some of the solution to move through the openings and form small particles on or above the outer surface of the active mesh 110. In some embodiments, active mesh 110 vibrates at from about 80 kHz to about 200 kHz upon electrical stimulation by an electrical current directed to the active mesh 110 by a controller board 107 and a mesh driver 103, although active mesh nebulizers having other vibrational frequencies are also within the scope of the present disclosure. In some embodiments, the active mesh 110 is made of titanium, platinum, or palladium, or alloys thereof or the like, or laminated layers of titanium, platinum, or palladium or the like, to produce the piezoelectric effect that results in mesh vibration and particle formation over the outer surface of the mesh in the mouthpiece interior volume. In some embodiments, the active mesh 110 is a stainless steel layer. In some embodiments, the active mesh 110 is a polymer layer with openings therethrough. Examples of polymer include polyimide, and the like. In some embodiments, the active mesh 110 includes nylon, polyethylene, and/or Teflon. In at least some embodiments, active mesh 110 is other than disc-shaped. In at least some embodiments, active mesh 110 is polygonal-shaped, rectangular-shaped, oval-shaped, elliptical-shaped, or the like.

According to some embodiments, the distribution of particle sizes in the plume of particles is configured to compensate for particles absorbing moisture during travel through the lung airways. As the particles pick up fluid from moisture in the lung, particle diameter increases. Particles which have a solution with a pH not equal to 7 have the pH adjust toward 7 by absorption of liquid from the lungs. When particles become too large, the likelihood of particles striking a lung surface prior to reaching an alveolar structure is increased.

Vial (or a vial assembly) 108 is located inside nebulizer body 106 and fits against a back side of a mouthpiece baseplate 112. A gasket 111 seals the juncture between the vial 108 and the backside of the mouthpiece baseplate 112 to prevent a solution 109 in the vial 108 from leaking. The vial 108 is sealed prior to connection to gasket 111 and mouthpiece baseplate 112 to prevent contamination, spillage, replacement, or removal of the solution 109 to ensure proper concentrations of pharmacological compound are delivered to a patient or user, and to avoid accidental over dosage of the patient or user by an unknown or unanticipated compound added to the vial before or during nebulizing of the solution 109 in the vial. In some embodiments, vial 108 is configured to hold from 1 to 10 milliliters (mL) of pharmacological compound solution, although other vial sizes, both larger than 10 mL, and smaller than 1 mL, are also within the scope of the present disclosure. In some embodiments, the vial is configured with a volume of about 6 mL and is configured to hold from 3 to 5 mL of pharmacological compound solution for the active mesh nebulizer 100. The volume of the vial 108 depends on the dosage, the frequency of doses, the value or concentration of the solution.

A controller board 107 in nebulizer body 106 regulates operation of the active mesh nebulizer 100. Controller board 107 includes a micro controller 116, a data storage 118, and a real time clock 120. Micro controller 116 is connected to a port 130 extending through an outer wall of the nebulizer body 106. In some embodiments, port 130 does not extend through the outer wall of the nebulizer body 106 and communicates data and/or power wirelessly with elements external to the nebulizer body 106. The micro controller 116 triggers a mesh driver 103 that drives the operation of the active mesh 110. In some embodiments, controller board 107 includes a wireless communication chip 122, an authentication controller 124, and/or a power regulator 126. In some embodiments, port 130 is a port configured to conduct power into a power supply 128 by use of controller board 107. In some embodiments, the power supply is a battery. In some embodiments, the power supply provides a voltage to the controller board 107 ranging from 1.5 volts to 9 volts. In some embodiments, the power supply is a lithium battery having a supply voltage ranging from 2.5 volts to 4.4 volts. In some embodiments, port 130 is configured to carry data between controller board 107 and an external computing device or a computer network adapter connected to the port 130. In some embodiments, port 130 is configured to conduct both power and data in order to promote configuration and/or operation of the active mesh nebulizer 100. In some embodiments, port 130 is a universal serial bus port or other power/data transfer port for computing devices known to practitioners of the art.

In some embodiments, a connected device, or an external computing device, sends instructions to the micro controller 116 in order to regulate operation of the active mesh 110, which are configured to determine performance parameters of the nebulizer. Performance parameters of the nebulizer include a start time of plume production, an end time of plume production, a duration of plume production, and a calculated volume of delivered solution, and a calculated amount of delivered pharmacological compound (e.g., a dose). In some embodiments, software instructions stored on the connected device, or external computing device, are configured to cause the active mesh nebulizer to transmit information about the nebulizer performance to the connected device or external computing device. Information about the nebulizer performance includes at least historical information about plume generation, pharmaceutical compounds, active mesh nebulizer performance characteristics, and the like. In some embodiments, the connected device or external computing device shares some or all information received from the active mesh nebulizer with the patient or user, or a third party such as a health care provider, a health care company, and/or a family member of the patient or user. In some embodiments, the external computing device is a tablet computer, a smartphone, a smart watch, a laptop computer, a desktop computer, or the like. In some embodiments, a communicative connection between the active mesh nebulizer and the external computing device is a wired connection over, e.g., a universal serial bus (USB) cable or another direct wired connection. In some embodiments, the communicative connection between the active mesh nebulizer and the external computing device is a wireless connection, as described below.

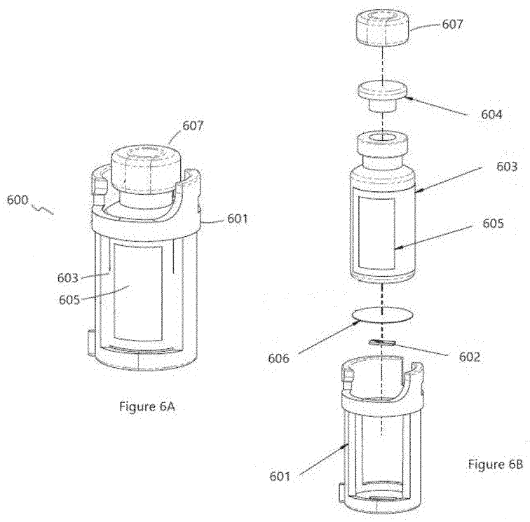

In some embodiments, vial 108 is configured with a vial identifier (identifier 113). In some embodiments, the identifier 113 is a barcode on a wall of the vial 108. In some embodiments, the barcode is printed directly on the vial. In some embodiments, the barcode is printed on a label that adheres to the wall of the vial. A printed label is used in some embodiments when elevated levels of reflectance are indicated to promote optical reading of a barcode. In some embodiments, the identifier 113 is a chip that performs an RFID (radio frequency identification) function, where the identifier provides information stored thereon, when requested, to the nebulizer 100. In some embodiments, the identifier 113 is an RFID chip located on the vial. In some embodiments, the identifier 113 is a crypto authentication chip with non-volatile memory (hereafter referred to as a crypto chip) located at a base of the vial (see, e.g., FIG. 6B, crypto chip 602). In some embodiments, the identifier 113 is a near field communications (NFC) chip located on the vial. In some embodiments, nebulizer 100 includes a reader 114 configured to capture information from the identifier 113 on a vial 108. In some embodiments, the reader 114 is an optical reader that scans a barcode-type identifier on a vial. In some embodiments, the reader is an RFID-type reader that requests and receives information stored on the identifier in the nebulizer body. In some embodiments, the reader includes at least one set of probes or pins which make electrical contact with the identifier 113. The reader reads information from the chip, and, in some embodiments, writes information to the chip. In some embodiments, the reader performs a write function to the identifier to indicate that the vial has been used and the full dose of medication has been delivered. In some embodiments, writing the information to the identifier 113 results in the vial being locked out from subsequent use in the nebulizer.

In some embodiments, authentication controller 124 is configured to record a biometric feature of a patient or user such as a fingerprint, iris image, retinal image, facial pattern, or other biometric identifying feature, or a password, passcode, electronic identifying code, or other security protocol or feature to restrict usage of the nebulizer 100 to approved or authenticated users, including users to whom the nebulizer 100 has been prescribed by a health care professional or other device supplier. In some embodiments, nebulizer 100 includes a fingerprint reader (not shown) to capture a fingerprint image for authentication. In some embodiments, a connected electronic device (such as a cell phone, tablet, smart watch, or other authenticated device) contains a biometric feature identifier such as a fingerprint reader, camera, or electronic password, passcode, electronic identifying code, or other security protocol interface to receive, from a user, the identifying authentication code and share, with the nebulizer 100, (or, the authentication controller 124 therein), the identifying authentication code or biometric feature information. In some embodiments, nebulizer 100 includes a biometric feature identifier such as a camera, or electronic password, passcode, electronic identifying code, or other security protocol interface to receive, from a user, the identifying authentication code and share, with the authentication controller 124 therein, the identifying authentication code or biometric feature information. In some embodiments, authentication controller 124 is configured to prevent activation of the active mesh by the micro controller until an authentication code or authorized biometric feature information has been received and verified by the authentication controller 124.

In some embodiments, the authentication controller performs authentication functions regarding a connected device, or an external computing device, which pairs, using an authentication protocol, to the nebulizer to reduce a likelihood of unauthorized use of the nebulizer when the connected device or external computing device is not present. In some embodiments, the connected device receives information from the nebulizer related to an identifier on the vial and compares information associated with the identifier on the vial to information related to the nebulizer and/or the external computing device, to confirm that the vial contains an anticipated and/or authorized pharmacological compound, that the vial contains an anticipated and/or authorized concentration of the pharmacological compound, that the vial is one of a number of anticipated and/or authorized number of vials linked, by the identifier, and/or information stored on at least the nebulizer and/or connected device (or external computing device) to the nebulizer to deliver the anticipated and/or authorized pharmacological compound to the patient or user. In some embodiments, a health care provider (such as a pharmacist, a physician, a physician's assistant, a nurse, or other authorized health care provider) inputs into the device the information to be accessed by the authentication controller. In some embodiments, the information is put on the connected device or external computing device.

In some embodiments, the nebulizer does not contain an on/off switch. In some embodiments, the nebulizer reads the identifier, verifies that the identifier is one of the approved identifiers associated with the nebulizer and any external computing device. In some embodiments, the nebulizer, after verifying that the identifier is on the list of approved identifiers, verifies that the nebulizer body is and remains closed. In some embodiments, when the identifier is verified to be approved, and when the nebulizer body is verified to be and remain closed, the micro controller 116, in conjunction with authentication controller 124, enables activation of the active mesh 110 upon a dosage request by a patient or user of the nebulizer.

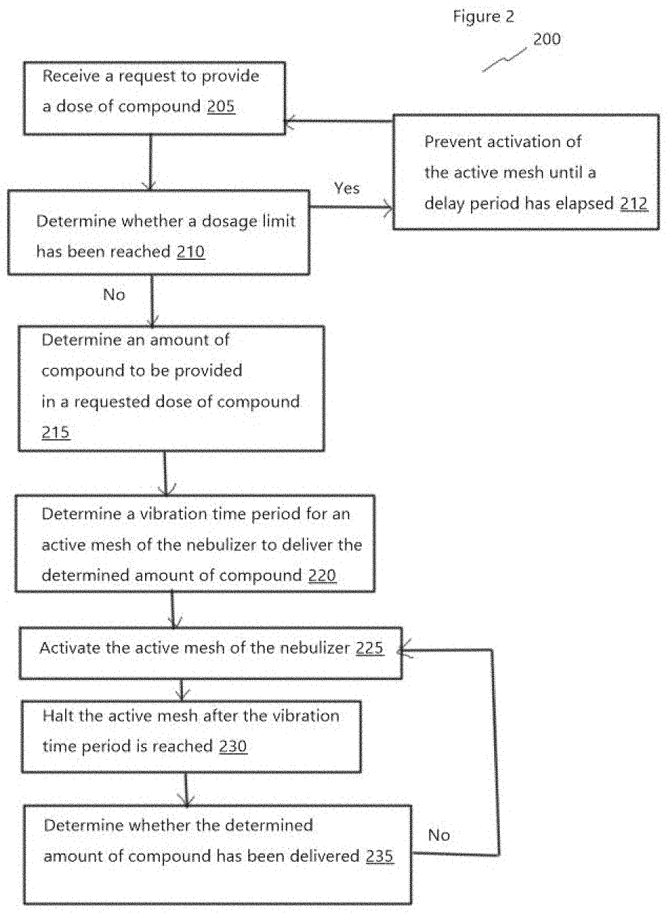

Controller board 107 includes the micro controller 116 and at least a non-transitory, computer-readable storage medium such as data storage 118 encoded with, e.g., storing, computer program code, e.g., a set of executable instructions. Data storage 118 is also encoded with instructions for executing a method of operating the nebulizer (FIG. 2). The micro controller 116 is electrically coupled to the data storage 118 via a bus 115 or other communication mechanism. The micro controller 116 also functions as an IO controller. Port 130 is also electrically connected to the micro controller 116 via the bus 115. Port 130 is configured to conduct communication and charging functions for the active mesh nebulizer 100. In some embodiments, port 130 conducts information via wireless communication protocols. In some embodiments, port 130 conducts data to an external computing device via a direct wired connection to an external computing device. In some embodiments, active mesh nebulizer 100 conducts data to an external computing device via a wireless connection to an external computing device. In some embodiments, port 130 conducts data to an external computing device over a wired network connection, and micro controller 116 and data storage 118 are capable of connecting to external elements or external computing devices via the network. In some embodiments, the micro controller 116 and the data storage 118 are configured to both send and receive data between the active mesh nebulizer 100 and an external computing device. The micro controller 116 is configured to execute the computer program code encoded in the data storage 118 in order to cause the nebulizer to be usable for performing a portion or all of the operations as described in the method.

In some embodiments, the micro controller 116 is a central processing unit (CPU), a multi-processor, a distributed processing system, an application specific integrated circuit (ASIC), and/or a suitable processing unit.

In some embodiments, the data storage 118 is an electronic, magnetic, optical, electromagnetic, infrared, and/or a semiconductor system (or apparatus or device). For example, the data storage 118 includes a semiconductor or solid-state memory, a magnetic tape, a removable computer diskette, a random access memory (RAM), a read-only memory (ROM), a rigid magnetic disk, and/or an optical disk. In some embodiments using optical disks, the data storage 118 includes a compact disk-read only memory (CD-ROM), a compact disk-read/write (CD-R/W), and/or a digital video disc (DVD).

In some embodiments, the data storage 118 stores the computer program code configured to cause controller board 107 to perform the method. In some embodiments, the data storage 118 also stores information needed for performing the method as well as information generated during performing the method, such as data and/or a set of executable instructions to perform the operation of the method.

In some embodiments, the data storage 118 stores instructions for interfacing with machines. The instructions enable micro controller 116 to generate instructions readable by the machines to effectively implement the method during a process.

Nebulizer 100 includes real time clock 120. The real time clock 120 is used when storing inhalation data, which includes the date, time, and duration of each inhalation. This data, along with the flow rate value, is used to calculate when the next inhalation is allowed to occur, and when the entire dose has been taken. This dosing data can be transmitted, via a smart phone etc., to the prescribing physician, and/or insurance provider.

Nebulizer 100 also includes a network interface, e.g., in the form of port 130, coupled to the micro controller 116. The network interface allows nebulizer 100 to communicate with a network, to which one or more other computer systems are connected. The network interface includes wireless network interfaces such as BLUETOOTH, WIFI, WIMAX, GPRS, or WCDMA; or wired network interface such as ETHERNET, USB, or IEEE-1394. In some embodiments, the method is implemented in two or more systems, and information is exchanged between different systems via the network.

FIG. 2 is a flow diagram of a method 200 of operating a nebulizer, e.g., nebulizer 100 (FIG. 1), in accordance with some embodiments. According to some embodiments of the present disclosure, the operations listed below are performed in the order described. In some embodiments, additional operations are performed in conjunction with the method to promote safe operation of a nebulizer to deliver doses of pharmacological compounds to a patient or user. In some embodiments, the operations listed below are performed in a different order than provided below, while still falling within the scope of the present disclosure to deliver a pharmacological compound to a patient or user.

Method 200 includes an operation 205, where a patient or user provides a request to a nebulizer and the nebulizer receives the request for a dose of a pharmacological compound. In some embodiments, the patient or user provides the request by pressing a button on the nebulizer body. In some embodiments, the patient or user provides the request by activating the nebulizer using a smartphone, computer tablet, or other electronic device that is communicatively paired with the nebulizer via the wireless communication module 122 on controller board 107. In some embodiments, the request is provided and received wirelessly using a wireless electronic communication such as WIFI™, Bluetooth™, or another wireless communication protocol. In some embodiments, the request is provided over a direct or wired connection to the micro controller 116 on controller board 107 through a communication port such as port 130.

Method 200 includes an operation 210, wherein the micro controller 116 determines whether a dosage limit has been reached for the patient or user at the time the patient or user makes the request to the nebulizer to provide a dose of the pharmacological compound. A dosage limit is a limitation on the total deliverable amount of pharmacological compound that is to be delivered to a patient or user during a dosage limitation period. Some dosage limits are related to short term (e.g., less than 12 hours) delivery periods for a pharmacological product. Some dosage limits are related to long term (e.g., greater than 12 hours, up to several days) delivery periods of a pharmacological product. In some embodiments, when determining whether a dosage limit has been reached, the micro controller 116 accesses data storage 118 to evaluate previous times of delivery of the pharmacological compound to the patient or user. In some embodiments, when determining whether a dosage limit has been reached, the micro controller 116 accesses data storage 118 to evaluate previous amounts of delivered pharmacological compound to the patient or user.

In some embodiments, the dosage limit is stored in the data storage 118. In some embodiments, the dosage limit is stored on vial 108 as part of vial identifier 113 and read by reader 114 for storage in data storage 118. In some embodiments, the dosage limit is received from a device external to nebulizer 100 via port 130.

When, based on at least one of the previous times of delivery of the pharmacological compound and the previous amounts of delivered pharmacological compound, the dosage limit of the pharmacological compound has been reached, method 200 proceeds to operation 212, wherein the active mesh is prevented from activating to deliver pharmacological compound until a delay period (e.g., a dosage delay period or dosage delay time) has elapsed. In some embodiments, the delay period is based on a calculated time in which a patient or user is expected to metabolize previously delivered pharmacological compound, including an amount of previously delivered pharmacological compound provided to the patient or user. In some embodiments, the delay period is based on a pre-determined time period associated with providing doses of the pharmacological compound. In some embodiments, the delay period is programmed into the nebulizer based on instructions from a health care provider or health care professional. In some embodiments, the delay period is based on an instruction provided to the nebulizer by the patient or user of the nebulizer. In some embodiments, the delay period is programmed into the nebulizer based on the type of pharmacological compound in the vial loaded into the nebulizer for the patient or user. In some embodiments, the delay period is a combination of one or more of the type of pharmacological compound, an instruction provided by a health care provider or health care professional, and a previously-delivered amount of the pharmacological compound. In some embodiments, operation 212 comprises a period in which the active mesh is not activated as opposed to preventing activation of the active mesh. After operation 212, the method proceeds to operation 205.

When, based on at least one of the previous times of delivery of the pharmacological compound and the previous amounts of delivered pharmacological compound, the dosage limit of the pharmacological compound has not been reached, method 200 proceeds to operation 215. Because the dosage limit has not been met, there is no triggering of a delay period before additional pharmacological compound delivery, and requests for dosing with the pharmacological compound are allowable by the nebulizer.

In operation 215, in preparation to delivering the pharmacological compound, the micro controller 116 determines an amount of compound to be provided via the active mesh in response to receiving the request received in operation 205. A determined amount of pharmacological compound to be provided in the requested dose is based on one or more of a time of the most recent dose of pharmacological compound, a quantity of the pharmacological compound provided in a most recent dose of the pharmacological compound, and the dosage limit of the pharmacological compound for the patient or user. In some embodiments, the determined amount of pharmacological compound is a full requested dose of compound because the size of a full requested dose (an initial dose size, or a standard dose size) does not exceed the dosage limit of the pharmacological compound. In some embodiments, the determined amount of pharmacological compound is a partial dose (or, a modified dose size), because a full dose of the pharmacological compound exceeds the dosage limit of the pharmacological compound. A dosage limit is based on a quantity of pharmacological compound delivered to a patient or user within a dosing time period. In some embodiments, the dosing time period is determined by a health care provider or professional. In some embodiments, the dosing time period is determined by the patient or user of the nebulizer. In some embodiments, the dosing time period is based on an average metabolism rate of the pharmacological compound by a patient or user.

Although previous discussion related to dose size calculation based on reductions in dose size, reductions in vibration time period of the active mesh, and smaller modified dose sizes in relation to delivering pharmacological compounds that approach a dosage limit of the pharmacological compound, aspects of the present disclosure also relate to determinations of increased vibration time of the active mesh, increased dose size (or repeated dosing in a short period of time), or larger modified dose sizes. Increases in dosing frequency, increased modified dose sizes, and increased vibration time period of the active mesh are most appropriate “early” in a dosage cycle, when a patient or user is not near to a dosage limit or dosage threshold of the pharmacological compound. In a non-limiting example, pain medication is delivered on demand to a patient or user upon a dosage request as often as a patient requests until the dosage threshold has been achieved, in order to address a patient's perceived pain levels. Should a patient continue to request pain medication at increased rates, the nebulizer is configured to provide information to medical providers about a number of times pain medication was requested, frequency of the requests, information about the medication being delivered, and medical providers are enabled to modify medications, modify limits of the medication delivery schedule or dosage limits, or initiate patient counseling or rehabilitative treatments to address addictive patterns of behavior before a patient becomes physically or mentally dependent on or addicted to the pain medication.

Method 200 includes an operation 220, wherein, based on a predetermined amount of pharmacological compound to be provided via the active mesh to the patient or user, the nebulizer determines at least one vibration time period (e.g., a calculated vibration time) of the nebulizer active mesh in order to deliver the pharmacological compound to the patient or user. The at least one vibration time period of the active mesh is determined based on one or more of the characteristics of the active mesh, the concentration of solute(s) in the solution of pharmacological compound in a vial in a nebulizer, a quantity of pharmacological compound to be provided, and whether or not the full requested dose is to be provided based on the dosage limit of the pharmacological compound.

Method 200 includes an operation 225, wherein the active mesh is activated in order to produce a plume of particles or droplets of a solution of a pharmaceutical product to be inhaled by a patient or user. In some embodiments, the nebulizer signals to the patient or user to begin inhaling through the mouthpiece 102 before the active mesh is activated to produce the plume of particles or droplets. One aspect of the present disclosure related to controlled and/or accurate dosage of the pharmacological product being provided to the patient or user is to generate an entirety of a plume of particles or droplets during a single inhalation event by the patient or user. The timing of the active mesh is controlled in order to produce a well-defined quantity of particles or droplets in the plume. In some embodiments, the timing of the active mesh is controlled to within +/−0.2 seconds when starting and stopping the mesh vibration to produce a plume of particles or droplets. In other embodiments, the timing of the active mesh is controlled to within +/−0.5 seconds or greater. In some embodiments, the period of time for generating a plume of particles is a plume generation interval. The total vibrational time of the active mesh is divided into a set of plume generation intervals to divide delivery of the mediation/pharmaceutical product into portions that can be inhaled by a user without interruption, where each plume generation interval corresponds to a period of time for generating one plume portion.

In some embodiments, after a vial 108 with solution is removed from the nebulizer 100, the active mesh is cleaned by activating the mesh with a vial of cleaning solution therein. In some instances, the cleaning solution is water. In some embodiments, the cleaning solution contains other antibacterial compounds for killing bacteria. In some embodiments, the active mesh is sterilized using UV light. Cleaning an active mesh eliminates biological contaminants that cause illness. For example, bacterial growth on an uncleaned active mesh is included in a plume of particles when no cleaning occurs, which contributes to elevated rates of respiratory illness in some patients or users of nebulizers. In some embodiments, the active mesh is cleaned on at least a daily basis. In some embodiments, the active mesh is cleaned on a weekly basis. In some embodiments, the active mesh is cleaned with hot water. After cleaning, the active mesh is allowed to air-dry. During cleaning, it is not recommended to bring solid objects into contact with the active mesh because the grid is prone to damage. For example, fingers, cotton swabs, cleaning cloths, and so forth, are the solid objects which are not recommended to come in contact with the active mesh because of the high likelihood of grid damage occurring.

According to some embodiments, the active mesh is vibrated for a cleaning period of at least 1 second, and up to 10 seconds, in order to remove contaminant materials from the active mesh surface, although cleaning periods longer than 10 seconds are also contemplated within the scope of the present disclosure. In some embodiments, the active mesh is vibrated when the solution in a vial 108 is in direct contact with one surface of the active mesh, in order to produce a cleaning plume, where the solution in the vial 108 flushes through the openings in the active mesh, to produce particles that are not for inhalation by a patient or user.

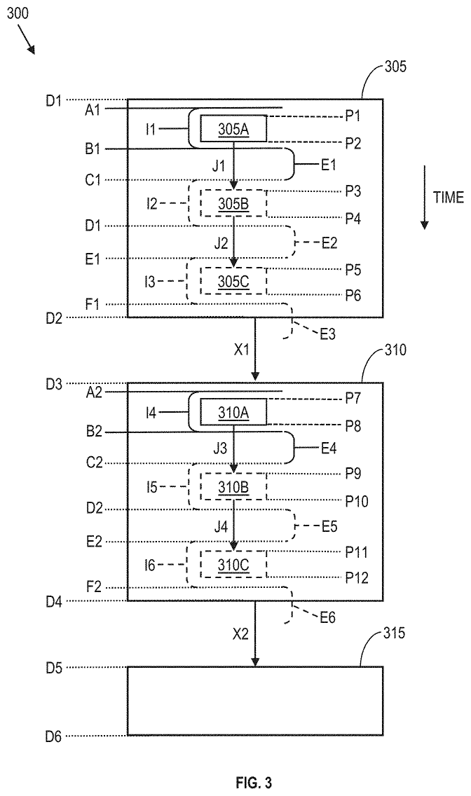

Method 200 includes an operation 230, wherein the active mesh is deactivated after providing some or all of a determined amount of the pharmacological compound. In some embodiments, the requested amount of the pharmacological compound is provided in a single activation period of the active mesh. In some embodiments, the requested amount of the pharmacological compound is provided over the course of several activation periods of the active mesh. Further discussion of the timing and duration of activation periods of the active mesh during delivery of a determined amount of pharmacological compound follows in the discussion of FIG. 3. In some embodiments, when the total vibration time period of an active mesh 110 to produce a plume of particles or droplets containing the determined amount of pharmacological compound exceeds a breath duration value (or an inhalation duration time), the total vibration time of the active mesh (see Plume Generation Interval, PGI, below) is divided into smaller time periods (smaller vibration time periods, or modified vibration times) that are smaller than a breath duration value (or an average breath duration of a patient or user) to avoid producing a plume of droplets or particles when the patient is not inhaling the plume into the lungs.

In an operation 235, the micro controller 116 determines whether the determined amount of pharmacological compound has been delivered. Determination of whether the determined amount of pharmacological compound has been delivered is made by using at least a computed vibrational time period of the active mesh, an actual or measured vibrational time of the mesh, and a calibration value of the active mesh 110 for producing a plume of particles of a solution in a vial in the nebulizer. In some embodiments, the determination further includes a calibration value for the total solute concentration of the solution in the vial, which has an impact on at least particle (or droplet) size in the plume of particles, and the mass flow of solution through the active mesh openings to make the plume of particles. When the determined amount of pharmacological compound has been delivered to the patient or user, the method continues to operation 210, wherein a determination is made about whether the dosage limit has been reached. When the determined amount of pharmacological compound has not been delivered to the patient or user, the method proceeds to operation 225 for at least one subsequent or second activation period of the active mesh 110 to deliver a remainder of the determined amount of pharmacological compound. In some embodiments, the total vibration time of the active mesh 110 is divided into uniform intervals to deliver the determined amount of pharmacological compound (and the at least one vibration time period of the active mesh 110 is uniform). In some embodiments, the total vibration time period of the active mesh 110 is divided into non-uniform intervals to deliver the determined amount of pharmacological compound (and not all of the at least one vibration time periods of the active mesh 110 are uniform). Uniform intervals are advantageous to help patients keep track of dosing progress and account for any missed plume inhalation time periods when determining total amounts of delivered pharmacological compound, or when reporting dosing received from the nebulizer to a health care provider or other third party. In some embodiments, non-uniform intervals are advantageous to deliver a maximum dosing of a pharmacological compound to a patient in the shortest amount of time or number of breaths.

FIG. 3 is a diagram of a nebulizer dosing program 300, in accordance with some embodiments. Nebulizer dosing program 300 includes a first dosing session 305 and a second dosing session 310. First dosing session 305 begins at a time D1 and ends at a time D2. A second dosing session 310 begins at a time D3 and ends at a time D4. A third dosing session 315 begins at a time D5 and ends at a time D6. Each dosing session includes at least one plume generation period, e.g., plume generation period 305A (or mesh activation period, see operations 220, 225, and 230 of method 200 in FIG. 2, above).

Each dosing session also includes at least one inhalation period, such as inhalation period I1 in first dosing session 305, and inhalation period I4 in second dosing session 310. In some embodiments, first dosing session 305 includes at least one additional inhalation period, such as inhalation periods I2 and I3, and second dosing session 310 includes at least one additional inhalation period, such as inhalation periods I5 and I6. Exhalation periods E1-E6 follow inhalation periods. For example, exhalation period E1 follows inhalation period I1 and precedes inhalation period I2. Some exhalation periods follow an inhalation period and a plume generation period, but are not considered part of a dosing session (see, e.g., exhalation period E3 after inhalation period I3, and exhalation period E6 after inhalation period I6).

Each inhalation period has an inhalation duration, and each exhalation period has an exhalation duration. In first dosing session 305, inhalation duration of inhalation period I1 extends from time A1 to time B1 [e.g., Inhalation Duration (ID)=Inhalation End Time (IET)−Inhalation Start Time (IST), see Table 1, below]. In first dosing session 305, exhalation duration of exhalation period E1 extends from time B1 to time C1 [e.g., Exhalation Duration (ED)=Exhalation End Time (EET)−Exhalation Start Time (EST), see Table 2, below]. First dosing session 305 includes plume generation period 305A. Plume generation period 305A extends from time P1 to P2 [e.g., plume duration (PD)=Plume End Time (PET)−Plume Start Time (PST), see Table 3, below]. In some embodiments, first dosing session 305 also includes at least one additional plume generation period, such as plume generation periods 305B and 305C.

When, in a dosing session, there are two or more inhalation periods and two or more plume generation periods, a pause (J) between adjacent plume generations periods extends from the end of one plume generation period and the start of a next plume generation period within the dosing session. For example, in first dosing session 305, pause J1 is between plume generation period 305A and plume generation period 305B, having a pause duration matching the time difference between time P3 and time P2 (e.g., P3−P2, see first dosing session 305, FIG. 3), and pause J2 is between plume generation period 305B and plume generation period 305C, having a pause duration matching the time difference between time P5 and time P4 (e.g., P5−P4).

In each dosing session, plume generation occurs for less time than the inhalation duration of the inhalation period of the patient or user. In some embodiments, plume generation both commences after the start of an inhalation period and ends before the end of the inhalation period. In some embodiments, plume generation begins before or at the same time as an inhalation period and ends before the inhalation period ends. In embodiments where plume generation begins before, or at the same time as, an inhalation period, the duration of any plume generation before an inhalation period is sufficiently short that the plume of generated particles is retained within the interior volume of the mouthpiece without flowing out of openings in the mouthpiece configured to allow air to pass between the interior volume and the exterior volume from the mouthpiece. In a preferred embodiment, the duration of a plume generation period is smaller than an inhalation period duration in order to increase the likelihood that the contents of the generated plume of particles or droplets is brought into the lungs without wasting portions of the plume by not being inhaled into the lungs.

In some embodiments of the nebulizer, the nebulizer indicates to a patient or user that an inhalation period should begin with commencement of a first signal or a first alarm (one or more of a vibration, a flashing or constant light, or, in the case of a user with visual impairment, a sound played by the nebulizer or the connected electronic device that triggers operation of the active mesh 110 to produce a plume of particles or droplets). Generation and/or cessation of signal or alarm s is indicated in operations 225 and 230 of method 200, described above. In some embodiments, the patient or user is informed that an inhalation period may end (because the plume production has stopped) with a second signal or second alarm, different from the first signal or first alarm. In some embodiments, the patient or user is informed that an inhalation period has ended with a cessation of the first signal or first alarm, which has remained continuous throughout the inhalation period. In some embodiments, the first signal or the second signal is a combination of one or more of a vibration, a flashing or constant light, or a sound played by the nebulizer or connected electronic device that triggers operation of the active mesh 110. In some embodiments, the first signal is one or more of a vibration, a light signal, or a sound played by the nebulizer or the connected electronic device, and the second signal is a different of one or more of a vibration, a light signal, or a sound played by the nebulizer or the connected electronic device. In some embodiments, signaling to indicate the commencement and ending of inhalation is repeated for each inhalation until a determined amount of pharmacological compound has been delivered by the nebulizer, up to a dosage limit of the pharmacological compound, or until a time threshold is reached at which point the nebulizer operation is halted.

In some embodiments, a connected device sends signals to start and/or stop vibration of the active mesh 110 to produce a plume of particles or droplets. In some embodiments, a connected device records the times and durations of active mesh activation, active mesh deactivation, calculated volumes of delivered pharmacological compound based on the recorded start times, stop times, and plume generation period durations for the nebulizer. In some embodiments, the connected device stores the information for subsequent transmission to a third party, including a health care provider or health-care company, or a family member of the patient or user.

A dosing session ends when the last plume generation period ends. Thus, in some embodiments, a dosing session end time coincides with the plume generation period ending time. Thus, in a non-limiting example, dosing session 305 ends at time D2, and time D2 may, in some embodiments, coincide with time P6. In some embodiments, time F1 also coincides with time P6.

A waiting period X1 extends from time D2 to time D3. A delay period X2 occurs when the patient must wait before receiving another dose and extends from time D4 to time D5 (the start of a third dosing session 315, see FIG. 3). Waiting period X1 is initiated by completion of a dosing session 305 and extends to the start of second dosing session 310, wherein the nebulizer is able to provide another dose of a pharmacological compound to a patient or user at any time. Delay period X2 is initiated by completion of second dosing session 310 and extends to the start of third dosing session 315, wherein the operation of the active mesh 110 in a nebulizer is blocked or halted because a patient or user has met a dosage limit of the pharmacological compound being delivered. A person of ordinary skill will recognize that other nebulizer dosing programs are also within the scope of the present disclosure while still meeting the medical treatment plans of a patient or user, or of satisfying a patient or user's at-will requests for nebulized doses of pharmacological compound, while still avoiding scenarios where an excess of compound is delivered to the patient or user within a prescribed time period.

Time periods, events, and durations for second dosing session 310 are labeled in a manner similar to dosing session 305, where the time labels A-F have terminal numbers incremented by 1, where pause identifiers (J) have terminal numerals incremented by 2, inhalation identifiers (I) and exhalation identifiers (E) have terminal numerals incremented by 3, and plume generation (P) time labels have terminal numerals incremented by 6, as compared to dosing session 305.

In some embodiments, the inhalation duration is an averaged value programmed into the nebulizer storage to regulate the duration of a plume generation period. In some embodiments, the inhalation duration is a value entered into the nebulizer storage by a health care professional, health care company, the patient or user, or a third party, to accommodate a patient or user's individual lung capacity or breathing pattern. In some embodiments, the plume generation period is equal, or evenly distributed, throughout a dosing session. In some embodiments, the plume generation period is unevenly distributed through a dosing session. A range of the inhalation duration is from about 2 seconds to about 10 seconds, although longer inhalation times are also within the scope of the present disclosure to accommodate patients with larger lung capacity or who are to be accommodated during nebulizer use with longer inhalation times for medical reasons (obstructed airways, which reduces the peak inhalation rate, likelihood of coughing or bronchospasm during inhalation, which would result in exhalation of some or all of the plume of particles or droplets before absorption by the lungs, and so forth). According to theory and belief, the small size of the particles produced by the active mesh 110, as described above, promotes facile entrainment of the particles deep into the lung structure, without particles impacting the lung tissues and triggering a cough reflex in the patient or user.

In some embodiments, the pause time (e.g., J1, J2, and so forth) is programmed into the nebulizer storage to regulate the overall pause duration between subsequent plume generations in a multi-plume dosing session. In some embodiments, the pause time is programmed by a patient or user, or a third party such as a physician, health care provider, health care company, or other third party to allow tuning of the pause time to accommodate individual comfort or breathing conditions of a patient or user to avoid wasting the plume of particles or droplets by coughing, bronchospasm, missing an opportunity to inhale a plume of particles or droplets, and so forth. For a nebulizer which produces a plume of particles ranging between 0.5 and 5 μm in diameter, the particles do not make contact with the lung tissue within the first three bronchial divisions of the lungs, there is no possibility of coughing by the user because the lower (4^{th} and greater) divisions of the bronchii do not have tissue which triggers coughing. In some embodiments, the nebulizer waits for a signal (button press, and so forth, on the nebulizer or a connected electronic device that regulates nebulizer operation) to the nebulizer from the patient or user before beginning a second plume generation period, or a dosing session, to ensure that the patient or user is prepared to inhale the plume of particles or droplets having the pharmacological compound therein. Thus, in some embodiments, the pause time (e.g., J1, J2, and so forth) is a variable time subject to user influence during operation of the nebulizer.

TABLE 1
Inhalation Periods

| | | | Elapsed time = |
|---|---|---|---|
| Event = | Start = Inhalation | End = Inhalation | Inhalation |
| Inhalation | Start Time (IST) | End Time (IET) | Duration (ID) |
| I1 | A1 | B1 | B1 − A1 |
| I2 | C1 | D1 | D1 − C1 |
| I3 | E1 | F1 | F1 − E1 |
| I4 | A2 | B2 | B2 − A2 |
| I5 | C2 | D2 | D2 − C2 |
| I6 | E2 | F2 | F2 − E2 |

TABLE 2
Exhalation Periods

| Event = | Start = Exhale | End = Exhale | Elapsed time = |
|---|---|---|---|
| Exhalation | Start Time (EST) | End Time (EET) | Exhale Duration (ED) |
| E1 | B1 | C1 | C1 − B1 |
| E2 | D1 | E1 | E1 − D1 |
| E3 | F1 | N/A | N/A |
| E4 | B2 | C2 | C2 − B2 |
| E5 | D2 | E2 | E2 − B2 |
| E6 | F2 | N/A | N/A |

TABLE 3
Plume Generation Periods

| Event = | Start = Plume | End = Plume | Elapsed time = |
|---|---|---|---|
| Plume | Start Time (PST) | End Time (PET) | Plume Duration (PD) |
| 305A | P1 | P2 | P2 − P1 |
| 305B | P3 | P4 | P4 − P3 |
| 305C | P5 | P6 | P6 − P5 |
| 310A | P7 | P8 | P8 − P7 |
| 310B | P9 | P10 | P10 − P9 |
| 310C | P11 | P12 | P12 − P11 |

TABLE 4
Pharmacological Compound Dosing Periods

| Event = | Dosing Event | Dosing Event | Dosing Event |
|---|---|---|---|
| Dosing Event | Start | End | Duration |
| 305 | D1 | D2 | D2 − D1 |
| 310 | D3 | D4 | D4 − D3 |
| 315 | D5 | D6 | D6 − D5 |

| Event = | | | |
|---|---|---|---|
| Wait or Delay | Start | End | Duration |
| W1 | D2 | D3 | D3 − D2 |
| Y1 | D4 | D5 | D5 − D4 |

FIG. 4 is a chart 400 of particle sizes in a plume of particles produced by an active mesh nebulizer disclosed by the present disclosure. In chart 400, the particles produced in the plume of particles are detected using a laser diode particle counter (ParticleScan PRO® model) mounted on a test chamber with a probe directed toward the chamber inner volume. The particle counter is 12 inches from the particle plume, and the test chamber was purged with HEPA filtered air gas. In chart 400, the fraction of particles larger than 5 micrometers is a small fraction of the total number of particles produced by the active mesh nebulizer disclosed by the present disclosure.

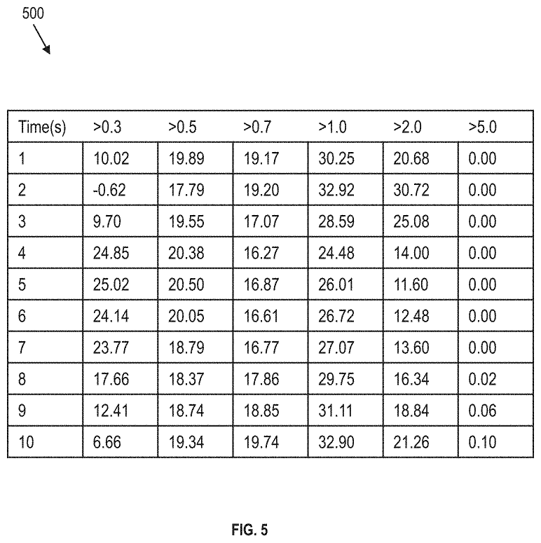

FIG. 5 is a table 500 of the average particle size information for each second of the first 10 seconds of active mesh nebulizer plume production by active mesh nebulizer disclosed herein. The values in columns of table 500 are averaged together and plotted to produce chart 400.

FIGS. 6A and 6B are diagrams of a vial assembly 600, in accordance with some embodiments. A vial assembly may be referred to as a cartridge or capsule. Vial assembly 600 includes a vial cage 601. In accordance with some embodiments, the vial cage 601 is constructed of plastic or another suitable material. In accordance with some embodiments, a crypto chip 602 is situated within the vial cage 601. An adhesive layer 606 is placed above the crypto chip 602. In accordance with some embodiments, the adhesive layer 606 is a very-high bond strength adhesive such as a permanent adhesive. The adhesive layer 606 securely maintains the crypto chip 602 in place within the vial cage 601. A vial 603 is adhered to the adhesive layer 606. In accordance with some embodiments, the vial 603 is a chip-resistant Corning Valor glass vial. The vial 603 contains a liquid (not shown). A stopper 604 closes the vial 603 and prevents leakage and contamination of the liquid within the vial 603. A tear-off seal 607 is fixed over the stopper 604. In accordance with various embodiments, the tear-off seal 607 is formed of crimped metal or an adhesive label. In accordance with some embodiments, the color of the tear-off seal 607 is used to identify the liquid within the vial 603. A label 605 is affixed to the vial 603. In accordance with some embodiments, the label 605 includes information regarding the liquid in the vial 603, including a 2D bar code and human-readable printing, containing information regarding the manufacturer of the medication, the medication, the volume of medication, the medication manufacturing lot number and the medication expiration date.

In accordance with some embodiments, the vial assembly 600 is packaged and sent to an ordering pharmacist. The pharmacist programs the patient's prescription information, such as the patient ID, medication, dosing amount, and dosing frequency, into a nebulizer. If the information contained in the crypto chip 602 matches the patient's prescription information, which the pharmacist programmed into the patient's nebulizer, the nebulizer dispenses the proper dose of medication at the proper intervals, until the prescribed number of doses have been dispensed. At which time, that vial assembly 600 is locked and no longer usable by the nebulizer to provide any further doses of medication. Each time the nebulizer dispenses a dose of medication, the date, time, and plume duration is written to the non-volatile memory of the active mesh nebulizer 100. This information is read by an external device and sent to the prescribing doctor and patient's insurance company for proof of proper dosing, enabling continued insurance coverage for that medication. In accordance with some embodiments, the nebulizer does not function without the application. The nebulizer tracks usage and writes the use of a dose back to the vial assembly and does not allow the vial assembly to be used again once the maximum allowed doses are consumed. This information is relayed back to the application via wired or wireless communication, e.g., such as Bluetooth or other communication method such as WIFI or Ethernet or the like.

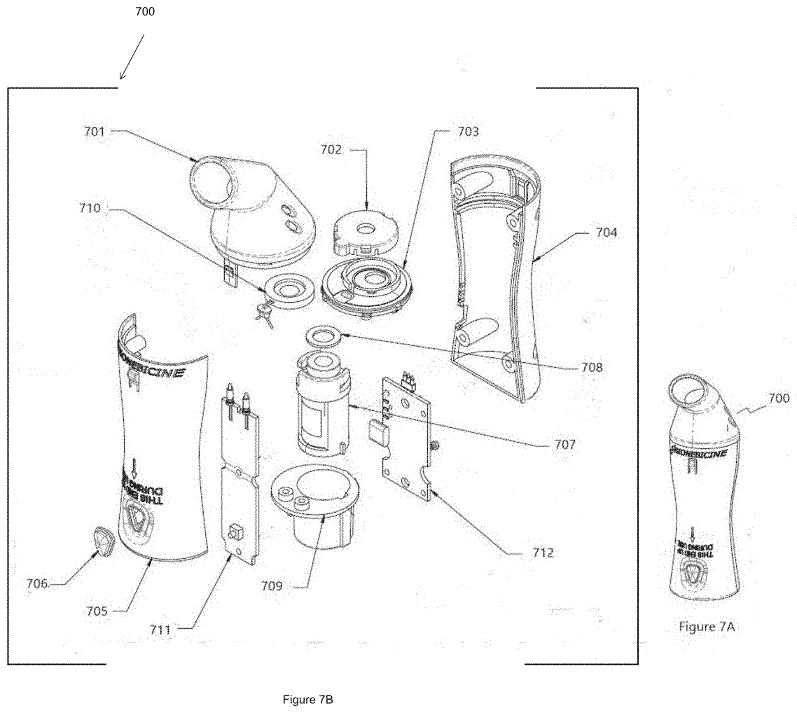

FIGS. 7A and 7B are diagrams of an active mesh nebulizer 700, in accordance with some embodiments. The active mesh nebulizer 700 includes a mouthpiece 701 situated on a handle formed by handle front 705 and handle back 704. A piezo-electric disc 710 is situated between a piezo disc nest 703 and a piezo disc cap 702. An on button 706 is situated on the handle front 705. A vial assembly 707, as described in FIG. 6A in connection with vial assembly 600, is placed within a vial receptacle 709 in the active mesh nebulizer 700. A soft washer 708 sits between the vial receptacle 709 and the piezo disc nest 703. A control circuit 712, corresponding to controller board 107 (FIG. 1), controls operation of the piezo-electric disc 710 as set forth in conjunction with method 200 (FIG. 2). The control circuit 712 also communicates with the crypto chip, such as crypto chip 602, within the vial assembly 707. A power circuit 711 is electrically connected to control circuit 712 in order to drive the piezo-electric disc 710 of the active mesh nebulizer 700.

When ready to receive a dose of medicine from the active mesh nebulizer 700, the patient turns the active mesh nebulizer 700 upside down, places the end of the mouthpiece 701 into their mouth, pushes and holds the on button 706 to begin operation of the active mesh nebulizer 700, and inhales a medication plume. The patient then releases the on button 706 to stop the operation of the active mesh nebulizer 700. When ready to take a next inhalation of medication, the patient repeats the process. After each inhalation, the duration of inhalation, the date, time and vial serial number are written to non-volatile memory in the control circuit 712 in the Nebulizer.

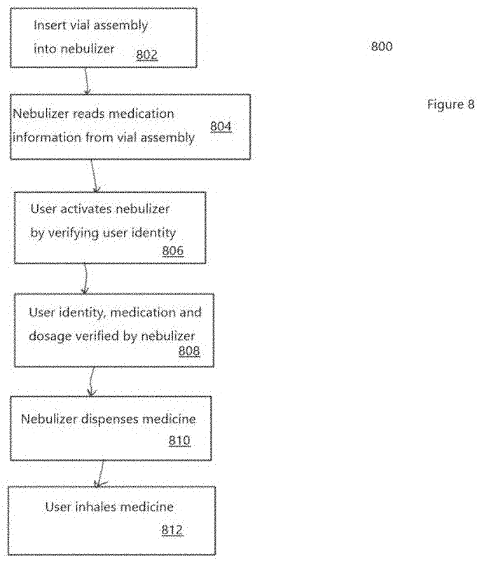

FIG. 8 is a flow diagram of a method 800 of operating an active mesh nebulizer, in accordance with some embodiments. The method 800 begins with step 802 when a user inserts a vial assembly cartridge into the nebulizer. In step 804, the nebulizer reads medication information from the vial assembly using a bar code reader, RFID, wired pin connector, or the like. In step 806, the user activates the nebulizer using an app on a smartphone or other device that verifies the user's identity, with a fingerprint reader, a PIN or another suitable method of authentication. In step 808, using the medication information and the user identity, the nebulizer verifies that the medication is authorized. In accordance with some embodiments, the nebulizer includes a conductivity sensor or pH sensor where the sensor extends into the vial volume on the liquid-side of the nebulizer mesh/grid. By measuring the conductivity of a fluid, the measured value is compared to a “calibrated” value of the fluid as prepared at the packaging plant. When there is a match, the nebulizer operates. When there is a mismatch, the nebulizer does not operate. This prevents the nebulizer from dispensing medicine when the original fluid has been replaced after the vial has been identified to the nebulizer, and before the active mesh 110 is activated. In step 810, if the medication is authorized, the nebulizer dispenses an appropriate dose of medicine. The user inhales the medication in step 812.

In accordance with some embodiments, communication between the nebulizer and smartphone app is used to authenticate the user and the medication as well as to control operation of the nebulizer. Security between the nebulizer and the smartphone app is performed using a secure protocol. Communication between the device and the phone is encrypted using proven protocols of encryption (minimum AES-CMAC—AES-128 via RFC 4493, which is FIPS-compliant, or ECDHE aka Elliptic Curve Diffie-Hellman aka P-256, which is also FIPS-compliant).

In accordance with some embodiments, security between the nebulizer and the vial assembly uses a crypto chip on the vial assembly to verify that the vial assembly is valid and then authorize its use in the nebulizer. This crypto chip provides the ability to store the number of uses or activations, which are limited via this same chip. The crypto chip, in accordance with some embodiments, is the SHA204A and a supplemental PIC16. The encryption protocol uses a 256-SHA hashing algorithm on this chip in hardware.

A hash with the secret key are compared between the nebulizer and the vial assembly and a match allows the usage of that vial assembly. A write once area on the crypto chip provides the ability to record the delivery of the full dose of medication, with the date and time of delivery to a user. Information related to the total number of uses of the nebulizer is stored in the non-volatile memory of the active mesh nebulizer.

The smartphone app prevents use of the nebulizer by an unauthorized user of the phone. The application opens and allows simple usage of the application, but upon activation of the ‘activate nebulizer’ command, the phone either requires a fingerprint or PIN or like identification of the user. Otherwise, the command to activate the nebulizer does not display.

In accordance with some embodiments, the nebulizer does not function without the application. The nebulizer tracks usage and writes the use of a dose back to the non-volatile memory in the nebulizer. This information is relayed back to the application via Bluetooth between the active mesh nebulizer and an authorized external computing device communicating with the active mesh nebulizer.

A single-insertion vial assembly allows the vial assembly to be inserted only one time, but when removed the vial assembly would not be able to be reinserted. In some embodiments, a single-insertion vial assembly is used to deliver a single large dose of a medication. In some embodiments, a single-insertion vial assembly is used to deliver multiple small doses of a medication or pharmacological compound before the vial assembly is removed. In some embodiments, the reinsertion of a used vial assembly is possible, but the nebulizer is programmed to not operate because the nebulizer recognizes the identifier associated with the vial assembly (a used vial assembly). A vial identifier has a unique serial number programmed therein at the chip manufacturer, and the active mesh nebulizer tracks vial usage using the unique serial number in the identifier 113 to prevent vial re-use.

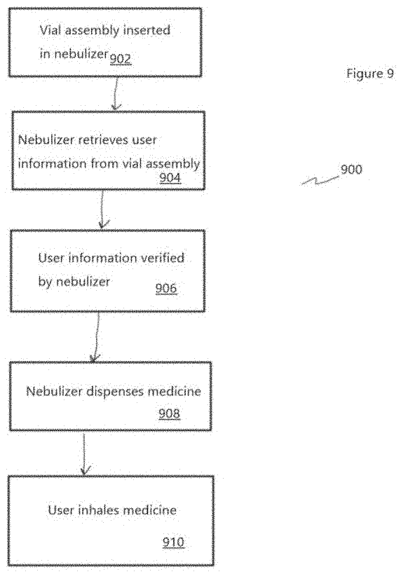

FIG. 9 is a flow diagram of a method 900 of operating an active mesh nebulizer, in accordance with some embodiments. The method 900 begins with step 902 when a user inserts a vial assembly cartridge into the nebulizer. In step 904, the nebulizer reads medication information from the vial assembly using a bar code reader, RFID, wired pin connector, or any other suitable data communication method. In step 906, using the medication information, the nebulizer verifies that the medication is authorized. In step 908, if the medication is authorized, the nebulizer dispenses an appropriate dose of medicine. The user inhales the medication in step 910.

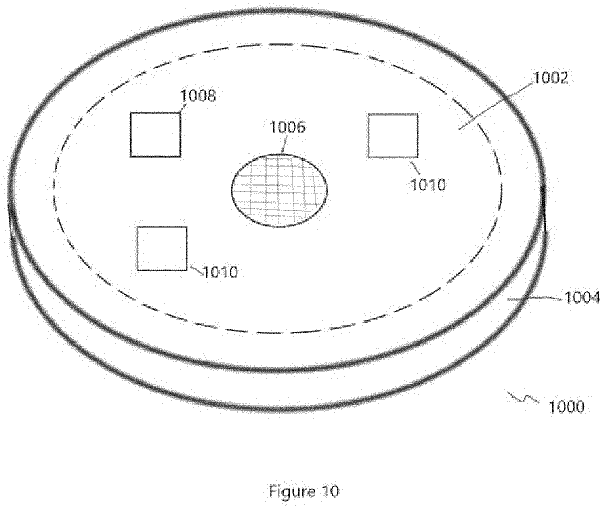

FIG. 10 is a diagram of a nebulizer mouthpiece baseplate 1000, in accordance with some embodiments. The nebulizer mouthpiece baseplate 1000, such as nebulizer mouthpiece baseplate 112 in FIG. 1 or piezo-electronic disc 710 in FIG. 7B, includes a base 1004 with a mounting surface 1002. At the center of the mounting surface 1002 is an active mesh 1006, such as in piezo-electronic disc 710 in FIG. 7B. The mounting surface 1002 includes a pH sensor 1008 and two conductivity sensors 1010, in accordance with some embodiments. In accordance with some embodiments, a nebulizer, such as nebulizer 100 in FIG. 1, includes a mouthpiece baseplate with a pH sensor 1008 and conductivity sensors 1010 that are flush with the mounting surface 1002. In accordance with some embodiments, the pH sensor 1008 and conductivity sensors 1010 extend into the vial volume on the liquid-side of the active mesh 1006. By measuring the conductivity and/or pH of a fluid, the measured value is compared to a calibrated value of the fluid as prepared at the packaging plant and read from vial assembly medication information. The comparison The comparison is made when the vial is initially connected to the nebulizer. In an embodiment, the comparison is made every time the vial is connected to the nebulizer. When there is a match between the measured conductivity and/or pH and the calibrated conductivity and/or pH value, the nebulizer is authorized to dispense medication. When there is a mismatch, the nebulizer displays an error message and does not dispense medication. This prevents the nebulizer from dispensing medicine with a different pH or conductivity when the original fluid has been replaced. On the reverse side of the nebulizer mouthpiece baseplate 1000, i.e., the opposite side from the mounting surface, the pH sensor 1008 and the conductivity sensors 1010 have connectors (not shown) to connect to the nebulizer controller board (e.g., controller board 107 in FIG. 1.

Matching the measured pH with stored information provides a safety check, so that incorrect or degraded medicine is not dispensed.

In accordance with some embodiments, pH sensor 1008 is a combination pH sensor having a reference electrode and a measuring electrode. The reference electrode is used to provide a stable signal, while the measuring electrode is designed to detect any changes that have occurred with the pH value due to adulteration, modification or degradation of the medicine. In accordance with some embodiments, pH sensor 1008 is a differential sensor having three electrodes, the third electrode of which is a metal ground electrode. Differential pH sensors reduce the risk of reference fouling.

Conductivity sensor 1010 measures the ability of the medicine solution to conduct an electrical current. The presence of ions in a solution allows the solution to be conductive: the greater the concentration of ions, the greater the conductivity. Conductivity sensor 1010 includes an electrode pair, to which a voltage is applied. The conductivity sensor 1010 measures the current flow and calculates the conductivity. In accordance with some embodiments, conductivity sensor 1010 is a 2-electrode conductivity meter including two parallel plates. An alternating current voltage is applied across the two electrodes, and the resistance between them is measured.

In accordance with some embodiments, conductivity sensor 1010 is a 4-electrode EC meter with an additional electrode pair. The outer electrodes are current electrodes to which an alternating current is applied; the outer electrodes are driven in the same manner as the 2-electrode conductivity sensor. In some embodiments, in-line conductivity electrodes are placed in the electric field of the current electrodes and measure the voltage with a high impedance amplifier. The current flowing through the outer electrodes and the solution is measured by the circuit. If the voltage across the inner electrodes and the current are known, the resistance and conductance is calculated. The 4-electrode conductivity sensor has negligible current flowing through the inner electrodes where the measurement is made. Therefore, no polarization effects occur which would otherwise influence the measurement. The 4-electrode conductivity sensor is also less sensitive to measuring errors through electrode fouling.

In accordance with some embodiments, the nebulizer includes a conductivity sensor 1010 and/or pH sensor 1008 where the sensor extends into the vial volume on the liquid-side of the active mesh 1006. By measuring the conductivity and/or pH of a fluid, the measured value is compared to a “calibrated” value of the fluid as prepared at the packaging plant.

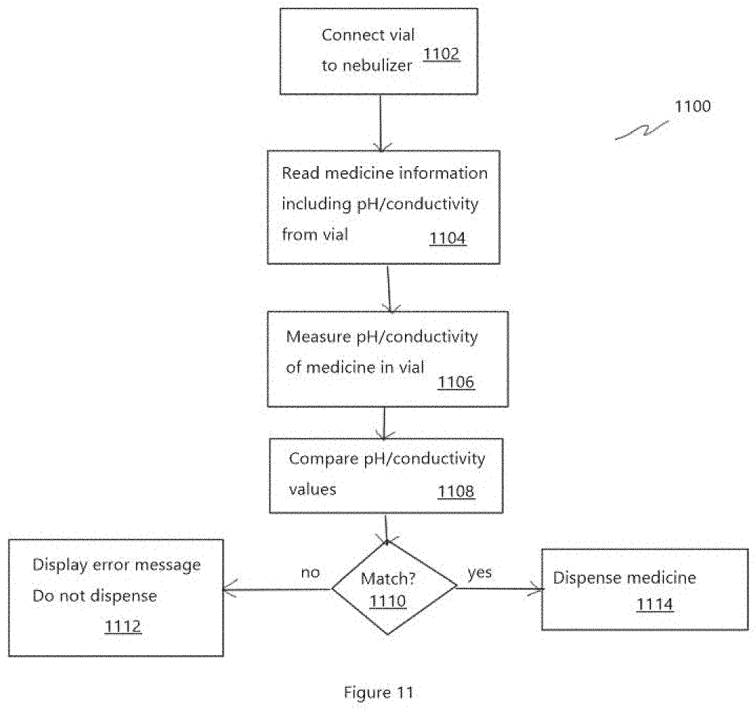

FIG. 11 is a flowchart of a method 1100 of operating an active mesh nebulizer, in accordance with some embodiments. The method 1100 begins with the step 1102 of connecting a medicine vial, e.g. vial 108 in FIG. 1 to the nebulizer, e.g. 100 in FIG. 1. At step 1104, the nebulizer reads medicine information including the pH/conductivity of the medicine from the vial identifier 113. At step 1106, using the pH sensor 1008 and the conductivity sensors 1010, the nebulizer measures the pH and conductivity of the medicine in the vial. At step 1108, the nebulizer compares the measured pH and conductivity values of the medicine in the vial with the pH and conductivity values read from the vial identifier 113. At decision step 1110, the nebulizer determines if the measured pH and conductivity values and the read pH and conductivity values match. In some embodiments, the measured conductivity and the expected conductivity matches within a predetermined tolerance in order for the nebulizer to dispense medicine. In some embodiments, the predetermined tolerance ranges from ±1% to ±3% at a given temperature, e.g., at 25 C. A measured conductivity that differs from the expected conductivity by more than the predetermined tolerance (e.g., from ±1% to ±3% at a given temperature, e.g., at 25 C) indicates that there is [1] an error in the temperature measurement, or [2] that the medicine has degraded, or [3] that there has been a human error (e.g., the wrong medicine is in the vial or nebulizer), or some other systematic or human error. In some embodiments, the read medicine pH value and the measured pH of the medicine solution match within a predetermined tolerance in order for the active mesh to be activated. In some embodiments, the predetermined tolerance ranges from +/−0.1 at a given temperature, e.g., at 25 C. A measured pH value that differs from the expected pH value by more than the predetermined tolerance (e.g., from ±0.1 at a given temperature, e.g., at 25 C) indicates that there is [1] an error in the temperature measurement, or [2] that the medicine has degraded, or [3] that there has been a human error (e.g., the wrong medicine is in the vial or nebulizer), or some other systematic or human error. If the values do not match, the process proceeds to step 1112 where medicine is not dispensed by the nebulizer 100. In at least one embodiment, medicine is dispensed only if both the measured pH and conductivity values match the corresponding read pH and conductivity values. In accordance with an embodiment, an error message is displayed. If the values do match, the nebulizer dispenses the medicine at step 1114.

The particles generated by the nebulizer are small (1.0-5.0 micrometers), so the particles infrequently impact with the lung tissue before the particles reach the alveoli. Particles that do impact the lung tissue can cause irritation and produce coughs in the user. Acidic or basic particles cause more irritation than particles with a neutral pH of 7.0. Because the nebulizer delivers particles with infrequent impacts, the pH of the solution does not cause irritation. Accordingly, the pH for a solution can be selected to be different from neutral to provide a longer lasting drug, i.e., be better absorbed, or the like.

For a single fluid having a given viscosity, by increasing the voltage applied to the nebulizer grid, the rate at which the liquid is converted into droplets increases. Liquids having different viscosities require different grid vibrational frequencies and/or supply voltages from the power supply (see power supply 128) in order to become plume particles. The voltage applied to the grid is regulated by a voltage regulating circuit in the nebulizer.

In accordance with some embodiments, the nebulizer is configured to adapt the grid voltage and/or frequency delivered to the active mesh 110 (or, grid) to produce a plume from liquids having different viscosities by adjusting the applied voltage and or active mesh vibrational frequency in response to an input providing information about the liquid identity and viscosity. In some embodiments, the active mesh nebulizer is configured to perform a frequency sweep to determine the current resonant frequency of the active mesh 110 or grid when producing the plume of particles.

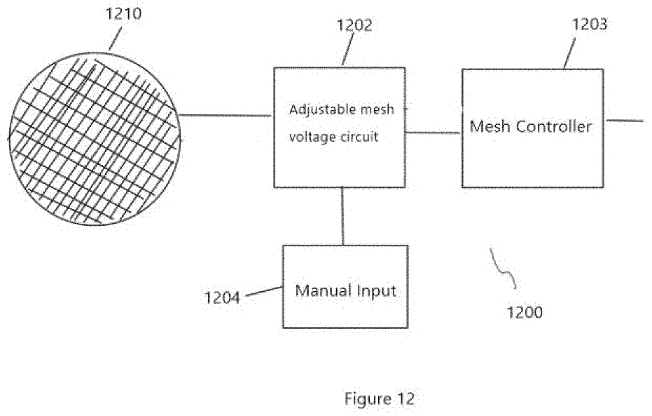

FIG. 12 is a diagram of an adjustable voltage active mesh nebulizer (adjustable voltage nebulizer 1200), in accordance with various embodiments. When the adjustable voltage nebulizer 1200 is operated, instructions are sent from the micro controller 1203 to an adjustable mesh voltage circuit 1202. The adjustable mesh voltage circuit 1202 provides a voltage to an active mesh 1210 in accordance with the instructions received from the micro controller 1203.

In accordance with some embodiments, the nebulizer receives an input (drug ID, viscosity number, or the like) from the vial assembly barcode or vial assembly security chip, or from the connected smartphone/tablet device, and dynamically adapts the grid voltage to produce a plume at a designated flow-rate, as part of the plume production calculation.

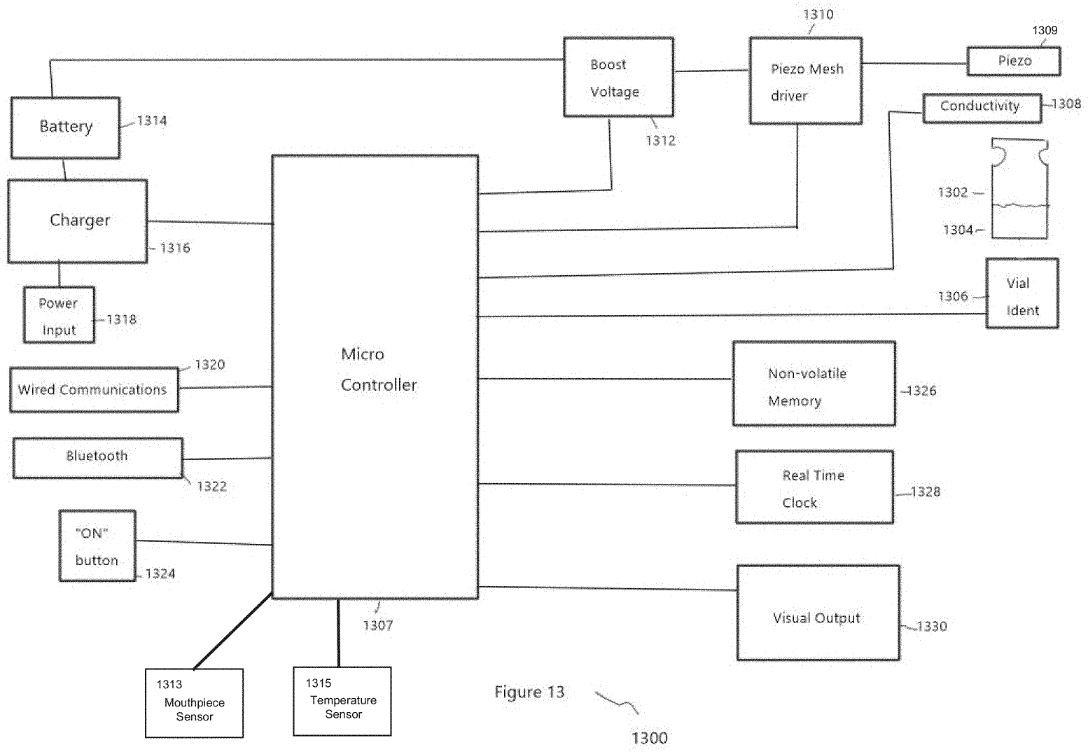

FIG. 13 is a block diagram of an active mesh nebulizer 1300, in accordance with an embodiment. The active mesh nebulizer 1300 is connected to a vial 1302 containing medicine 1304. The vial 1302 includes a vial identifier (not shown). The vial identifier is similar to vial identifier 113 in FIG. 1 and includes information about the medicine 1304 contained in the vial 1302. In accordance with an embodiment, the vial identifier includes information about the patient or user of the nebulizer. A vial identifier reader 1306 reads the vial identifier of the vial 1302. In some embodiments, the vial identifier reader 1306 emits an electromagnetic pulse which interacts with the vial identifier 113, and the vial identifier transmits a response pulse containing information about the medicine 1304, the patient or user, and other information related to provide patient care (see also the description of vial 113, above). The vial identifier reader 1306, similar to the reader 114 in FIG. 1, is connected to a micro controller 1307. The vial identifier reader 1306 communicates the vial identifier to the micro controller 1307.

The micro controller 1307, similar to the processor 116, is programmed to receive the vial identifier from the vial identifier reader 1306. The vial identifier includes the expected conductivity of the medicine 1304. When the vial 1302 is inverted so that the medicine 1304 contacts a conductivity sensor 1308, the conductivity of the medicine 1304 is measured and the measured conductivity is compared to the expected conductivity of the medicine obtained from the vial identifier. Upon determining that the measured conductivity and the expected conductivity match, the micro controller 1307 communicates with a piezoelectric mesh driver 1310 with instructions to energize the piezoelectric mesh 1309, similar to active mesh 110 in FIG. 1. In some embodiments, the measured conductivity and the expected conductivity matches within a predetermined tolerance in order for the micro controller 1307 to communicate with the piezoelectric mesh driver 1310. In some embodiments, the predetermined tolerance ranges from +/−1-3% at a given temperature, e.g., at 25 C. The micro controller 1307 communicates the dosage voltage to a boost voltage circuit 1312. Boost voltage circuit 1312 is a boost converter (also referred to as a step-up converter) which steps up the voltage from its input, i.e., from battery 1314, to the output connected to the piezo mesh driver 1310. The boost voltage circuit 1312 provides the dosage voltage to the piezoelectric mesh driver 1310.

In some embodiments, the piezoelectric grid flow-rate is a function of voltage applied to the active mesh 1310. Particle production rates using active mesh nebulizers increase with larger voltages applied to the active mesh. This increase in created particles follows a predictable rate of increase with the increase in piezoelectric grid voltage. The mesh controller selects the supply voltage based on the desired flow rate of the medication to dispense medication to the patient. A faster delivery rate provides more particles per second. Selecting an appropriate grid voltage generates the appropriate dose of medicine based on the type of medicine supplied in the vial 1302 and identified by the vial identifier (not shown).

The boost voltage circuit 1312 receives power from a battery 1314. The battery 1314 is connected to a charger 1316, similar to power regulator 126 in FIG. 1. The charger is connected to a power input 1318, similar to power supply 128 in FIG. 1. The charger 1316 is controllably connected to the microcontroller 1307.

The micro controller 1307 receives instructions and data from wired communication connections 1320, similar to port 130 in FIG. 1, and Bluetooth 1322, similar to wireless communication chip 122 in FIG. 1. The micro controller 1307 is connected to an “on” button 1324 to put the nebulizer in an “on” or powered state. The micro controller 1307 is connected to non-volatile memory 1326 to hold programming and data, similar to data storage 118 in FIG. 1. The micro controller 1307 is connected to a real time clock 1328 to provide an accurate measurement of the passage of time. The micro controller 1307 is connected to a visual output 1330 to indicate the “on” state or to indicate an “error”.

In active mesh nebulizer 1300, a mouthpiece sensor 1313 electrically connects to the micro controller 1307 in order to indicate the presence or absence of the mouthpiece on the active mesh nebulizer. When the mouthpiece is absent, the mouthpiece sensor 1313 sends a signal to the micro controller 1307, and the micro controller 1307 prevents activation of the piezoelectric mesh 1309 to avoid wasting medication which is not directed through the mouthpiece during an inhalation.

In active mesh nebulizer 1300, a temperature sensor 1315 is electrically connected to the micro controller 1307 in order to provide a temperature measurement for calibrating the measurements of sensors in the active mesh nebulizer. In some embodiments, the temperature sensor 1315 is used to provide a temperature measurement for calibration of a pH measurement by a pH sensor. In some embodiments, the temperature sensor 1315 is used to provide a temperature measurement for calibration of an electrical conductivity measurement by a conductivity sensor, e.g., conductivity sensor 1308. In some embodiments, the temperature sensor 1315 measures air temperature in the body of the active mesh nebulizer 1300. In some embodiments, the temperature sensor 1315 measures the temperature of the medicine 1304 in the vial 1302. In some embodiments, the temperature sensor 1315 measures the temperature of the vial 1302. In some embodiments, there are separate temperature sensors for the body of the nebulizer, the medicine, and the vial.

Temperature measurements increase the accuracy of measuring pH and electrical conductivity of medicine 1304 in the vial 1302. When temperature measurements are provided to the micro controller 1307, the predetermined thresholds for pH and electrical conductivity are smaller than when no temperature measurements are provided to the micro controller 1307.

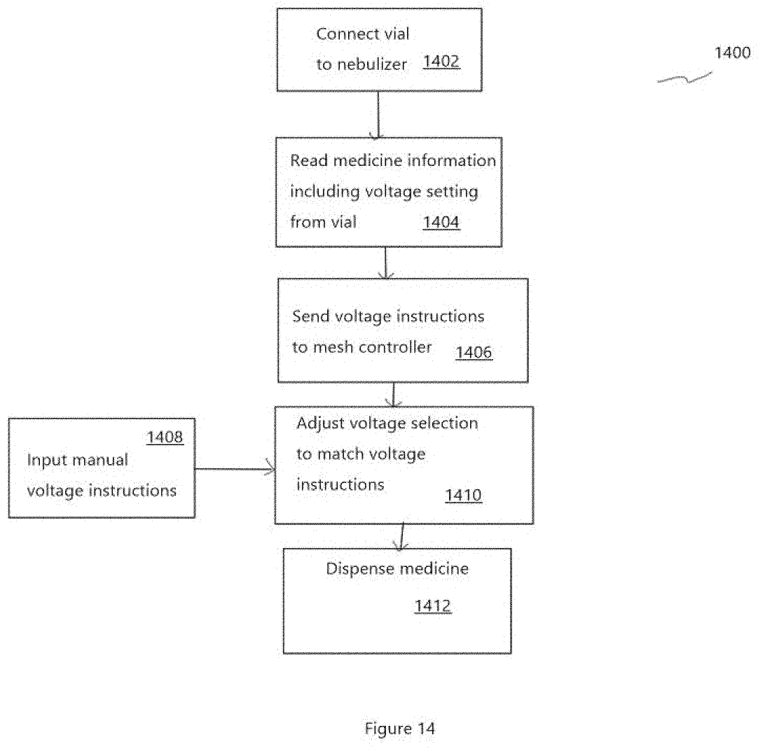

FIG. 14 is a flowchart of a method 1400 of operating an active mesh nebulizer, in accordance with some embodiments. At step 1402, a vial of medicine, e.g., vial 108 is connected to nebulizer 100 (FIG. 1). At step 1404, the nebulizer reads medicine information including the appropriate flow rate setting from the vial identifier 113. At step 1406, the nebulizer sends voltage instructions to the mesh driver (e.g., mesh driver 103 of FIG. 1). At step 1410, the micro controller adjusts the voltage selection to match the received flow rate instructions. At step 1410, the nebulizer dispenses the medicine at the selected flow rate setting.

A method of using a nebulizer includes connecting a medicine vial containing a medicine solution to the nebulizer and reading a medicine pH value from the medicine vial. The pH of the medicine solution is measured and compared with the medicine pH value. When the medicine pH value and the measured pH of the medicine solution match, an active mesh is activated to produce a plume of particles of a medicine solution after a beginning of an inhalation. The active mesh is deactivated to halt production of the plume of particles during the inhalation. The pH is measured by a pH sensor. The expected medicine pH value is read from a vial identifier. When the expected medicine pH value and the measured pH of the medicine solution do not match, the nebulizer displays an error message. In some embodiments, the expected medicine pH value and the measured pH of the medicine solution match within a predetermined tolerance in order for the active mesh to be activated. In some embodiments, the predetermined tolerance ranges from +/−0.1 pH units at a given temperature, e.g., at 25 C. After deactivating the active mesh, a determination is made whether an initial dose size has been delivered by the nebulizer; if not, reactivation of the active mesh is allowed, to deliver a further plume of the solution during a further inhalation.

A method of using a nebulizer includes connecting a medicine vial containing a medicine solution to the nebulizer and reading medicine information including a medicine conductivity value from the medicine vial. The conductivity of the medicine solution is measured and compared with the medicine conductivity value. When the medicine conductivity value and the measured conductivity of the medicine solution match, an active mesh is activated to produce a plume of particles of the medicine solution after a beginning of an inhalation. The active mesh is deactivated to halt production of the plume of particles during the inhalation. The conductivity is measured by a conductivity sensor. The medicine conductivity value is read from a vial identifier. In at least one embodiment, the vial identifier is a crypto chip. In at least one embodiment, the vial identifier is part of a crypto chip. In at least one embodiment, the vial identifier is stored on a crypto chip. When the medicine conductivity value and the measured conductivity of the medicine solution do not match, the nebulizer displays an error message. After deactivating the active mesh, a determination is made whether an initial dose size has been delivered by the nebulizer; if not, reactivation of the active mesh is allowed, to deliver a further plume of the solution during a further inhalation.

A method of using a nebulizer includes connecting a medicine vial containing a medicine solution to the nebulizer and reading medicine information including a medicine flow rate value from the medicine vial. An active mesh is activated at the medicine flow rate value to produce a plume of particles of the medicine solution after a beginning of an inhalation. The active mesh halts production of the plume of particles during the inhalation to ensure that all medicine generated as inhalable droplets is inhaled, rather than lost (e.g., to ambient air) outside of an inhalation. The medicine flow rate value is read from a vial identifier. The medicine flow rate value corresponds to a discrete voltage value applied by the nebulizer to the active mesh.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of using a nebulizer, comprising:
connecting a medicine vial containing a medicine solution to the nebulizer;
reading a medicine pH value from the medicine vial;
measuring a pH of the medicine solution;
comparing the medicine pH value with the measured pH of the medicine solution;
when the medicine pH value and the measured pH of the medicine solution match, activating an active mesh to produce a plume of particles of a medicine solution after a beginning of an inhalation; and
deactivating the active mesh to halt production of the plume of particles during the inhalation.

2. The method of claim 1, wherein the measured pH is measured by a pH sensor.

3. The method of claim 1, wherein the medicine pH value is read from a vial identifier.

4. The method of claim 1, wherein the medicine pH value is a predetermined range of pH values.

5. The method of claim 1, wherein when the medicine pH value and the measured pH of the medicine solution do not match, the nebulizer displays an error message.

6. The method of claim 1, further comprising determining, after deactivating the active mesh, whether an initial dose size has been delivered by the nebulizer, and allowing reactivation of the active mesh to deliver a further plume of the medicine solution during a further inhalation.

7. An active mesh nebulizer, comprising:
an active mesh configured to produce a plume of particles of a solution in contact with the active mesh;
a micro controller configured to activate and deactivate the active mesh,
a medicine information reader for reading medicine information including pH and conductivity values for the solution;
a pH sensor connected to the micro controller configured to measure the pH of the solution wherein the micro controller allows activation of the active mesh when the measured pH is within a predetermined range of the pH value for the solution; and
a conductivity sensor connected to the micro controller configured to measure the conductivity of the solution wherein the micro controller allows activation of the active mesh when the measured conductivity is within a predetermined range of the conductivity value for the solution.

8. The nebulizer of claim 7, wherein the conductivity sensor includes two electrodes.

9. The nebulizer of claim 7, wherein the medicine information is read from a vial identifier.

10. The nebulizer of claim 9, wherein the vial identifier is a crypto authentication chip with non-volatile memory.

11. The nebulizer of claim 7, wherein the pH sensor is mounted on a mouthpiece baseplate and extends into the solution.

12. The nebulizer of claim 7, wherein the conductivity sensor is mounted on a mouthpiece baseplate and extends into the solution.

13. The nebulizer of claim 7, wherein the pH sensor is a differential pH sensor.

14. A method of using a nebulizer, comprising:
connecting a medicine vial containing a medicine solution to the nebulizer;
reading medicine information including a medicine flow rate value from the medicine vial,
activating an active mesh at the medicine flow rate value to produce a plume of particles of the medicine solution after a beginning of an inhalation; and
deactivating the active mesh to halt production of the plume of particles during the inhalation.

15. The method of claim 14, wherein the medicine flow rate value is read from a vial identifier.

16. The method of claim 14, wherein the medicine flow rate value corresponds to a discrete voltage value applied by the nebulizer to the active mesh.

17. The method of claim 14, wherein the activation of the active mesh is restricted to approved or authenticated users by means of an authentication code or biometric feature information.

18. The method of claim 14, further comprising receiving a personal flow rate value from an input device and activating the active mesh at the personal flow rate value.

19. The method of claim 18 where the input device is a smart phone.

20. The method of claim 18 further comprising a discrete voltage value applied by a voltage stepper to achieve various desired flow rates.
